(19) United States
(12) Patent Application Publication
(10) Pub. No.: US 2011/0318333 A1
(43) Pub. Date: Dec. 29, 2011

(54) NOVEL NON-TOXIC COMPOSITION AND METHOD OF USING SUCH FOR TREATING A DEGENERATIVE OR AN IMMUNE SYSTEM-RELATED DISEASE

(76) Inventors: Robert GAUTHIER, Dunvegan (CA); Lee H. LORENZEN, Irvine, CA (US)

(21) Appl. No.: 13/228,213

(22) Filed: Sep. 8, 2011

Related U.S. Application Data
(63) Continuation of application No. 11/987,196, filed on Nov. 28, 2007, now abandoned.
(60) Provisional application No. 60/899,656, filed on Feb. 6, 2007.

(51) Int. Cl. A61K 39/395; A61P 43/00; A61P 37/00

(52) U.S. Cl. 424/130.1

(57) ABSTRACT

There is provided a non-toxic composition and method of using such for treating a degenerative or an immune-related disease. The non-toxic composition comprises a sprouted grain composition comprising digestive enzymes, a clustered water composition, and a substantially undenatured whey protein composition.

### BACKGROUND OF THE INVENTION

This application claims the benefit of U.S. Provisional Patent Application No. 60/899,656 filed Feb. 6, 2007, which is hereby incorporated by reference.

1. Field of the Invention

This application describes a novel non-toxic composition and method of using such for treating a degenerative or an immune system-related disease. More particularly, this application describes a composition comprising a whey protein composition, a clustered water composition and a sprouted grain composition, and a method of using such for treating a degenerative or an immune system-related disease.

2. Description of the State of the Art

The following review of the state of the art is merely provided to aid in the understanding of the present invention and neither it nor any of the references cited within it are admitted to be prior art to the present invention.

### 2.1. Toxicity of Modern Pharmaceuticals

Modern pharmaceuticals often have undesired side effects and/or are toxic when used chronically. Such toxicity is particularly exemplified with individuals with immune system-related diseases which undergo therapy with modern pharmaceuticals.

For example, approximately 30-50% of human immunodeficiency virus (HIV)-infected individuals that undergo an anti-viral tri-therapy (HAART) eventually may develop lipodystrophy syndrome (HALS), which is acquired fat redistribution, that is, peripheral fat loss often accompanied by central fat accumulation. HALS is characterised by impaired glucose and lipid metabolism and other risk factors for cardiovascular disease (Haugaard, Expert Opin. Drug Metab. Toxicol. 2006, June; 2(3): 429-45).

In another example, Hepatitis C virus (HCV) is one of the main causative agents of chronic viral hepatitis. Chronic hepatitis C can progress to cirrhosis and eventually to hepatocellular carcinoma over a period of 20 to 30 years (Levent et al., J. Transl. Med. 2006, 4: 25). Anemia is associated with antiviral therapy in chronic hepatitis C (Hung et al., Liver Int. 2006, 26(9): 1079-86). Manns et al. have suggested that there is a limited efficacy in treatment of patients with hepatitis C virus (HCV) genotype 1 with the current standard therapy (pegylated interferon alpha and ribavirin) and the side effect profile necessitates the development of new therapeutic approaches (Manns et al., Gut. 2006, 55(9): 1350-9).

In a further example, Central nervous system (CNS) neurotoxicity is the dose-limiting side effect of treatment for systemic and CNS neoplasms (cancer) (Schiff and Wen, Hematol. Oncol. Clin. North Am. 2006, 20(6): 1377-98). Furthermore, chemotherapy for cancer is an intense and cyclic treatment associated with number of side-effects, such as psychological distress, anxiety and depression (Pandey et al., World J. Surg. Oncol. 2006, 26; 4:68).

These modern pharmaceutical-induced problems individually or taken as a whole may contribute to the overall fatigue and overall body weakening, which may impair the organism's ability to recover from an illness and/or responsiveness to a treatment.

Therefore, there is a need for a non-toxic composition and method of using such for treating an immune system-related disease, which will not have the above toxic effects.

### 2.2. Whey Protein Administration; Treatment of HIV-Infected Individuals

HIV infection and AIDS deplete glutathione (GSH) levels in the infected host (Sbrana et al. Electrophoresis. 2004, 25(10-11): 1522-9; Gil et al. Pharmacol Res. 2003, 32(3): 217-24; Ogunro et al., Niger Postgrad. Med. J. 2006, 13(1): 1-5).

Fraternale et al. teach that in an AIDS murine model, HIV-infected hosts can significantly reduce proviral content in infected organs including the brain (Fraternale et al., J. Acquir. Immune Defic. Syndr. 1999, 1; 21(2): 81-9), or reduces the progression of murine AIDS by maintaining high levels of intracellular GSH (Fraternale et al., Eur. J. Clin. Invest. 2001, 31(3): 248-52).

Micke et al. teach that administration of undenatured whey protein to an HIV-infected human elevates glutathione levels. However, body weight, T-cell counts, and other clinical parameters did not change in that study (Micke et al. Eur J Nutr. 2002, 41(1): 12-8; Micke et al. Eur. J. Clin. Invest. 2001, 31(2): 171-8).

Moreno et al. teach that administration of undenatured whey concentrate to children with rapidly progressive AIDS induce a significant median increase of 16.14 mg/dl (p=0.018) in erythrocyte glutathione levels. However, the TCD4/CD8 lymphocyte ratio showed a non-significant increase (Moreno et al. J. Trop. Pediatr. 2006, 52(1): 34-8).

U.S. Pat. No. 5,451,412 (Bounos et al. filed Jun. 29, 1993) relates to a whey protein composition comprising undenatured whey protein concentrate and a method of producing such.

U.S. Pat. No. 5,451,412 also teaches a method for improving the humoral immune response in mouse and for increasing the rate of synthesis, rate of replenishment and concentration levels of glutathione in the mouse organs, by using said whey protein composition. However, Because the whey protein concentrate was tested in comparison to a limited number of proteins, U.S. Pat. No. 5,451,412 reports that it cannot be ascertained whether the enhancement of the humoral immune response observed in five unrelated strains of mice fed a whey protein diet, was due to a real immunoenhancement, in absolute terms, by whey protein feeding or immuno-depression by the other food proteins tested.

Thus, it remains unclear whether administration of undenatured whey protein composition alone has any therapeutic benefit on HIV-infected humans. It also remains unclear whether some individuals may be substantially unresponsive to the undenatured whey protein administration due to inefficient assimilation of the undenatured whey protein composition.

### 2.3. Whey Protein Administration; Treatment of Hepatitis Virus-Infected Individuals

GSH decreases in the blood serum of Hepatitis C virus (HCV)-infected patients and this decrease has been suggested as being a factor underlying the resistance to interferon therapy, fostering HCV replication and may thus represent a biological basis for GSH replacement therapy (Barbaro et al., Am. J. Gastroenterol. 1996, 91(12): 2569-73).

Pak et al teach that acute Hepatitis B in adults, as well, diminishes intracellular GSH levels (Pak et al., Klin. Med. Mosk. 1991, 69: 54-57).

Cemek et al teach that Hepatitis A virus induces oxidative stress in children with Hepatitis A and reduces GSH levels (Cemek et al., World J Gastroenterol. 2006, 14; 12(38): 6212-5).

Watanabe et al. teach that administration of an undenatured whey protein concentrate to an Hepatitis virus (B or C)-infected individual elevates glutathione levels in Hepatitis B-infected patients, but that there were no significant changes for Hepatitis C-infected patients (Watanabe et al., J. Med. 2000, 31(5-6): 283-302).

Tanaka et al. teach that administration of bovine lactoferrin, a protein subfraction of whey protein, to patients with chronic Hepatitis C induce a decrease in serum alanine transaminase and HCV RNA concentrations in 3 (75%) of 4 patients with low pretreatment serum concentrations of HCV RNA. However, 7 patients with high pretreatment concentrations showed no significant changes in these indices (Tanaka et al., Japan J. Cancer Res. 1999, 90(4): 367-71).

Thus, it remains unclear whether administration of undenatured whey protein composition alone has any therapeutic effect on Hepatitis virus-infected individuals. It also remains unclear whether some individuals may be substantially unresponsive to the undenatured whey protein administration due to inefficient assimilation of the undenatured whey protein composition.

### 2.4. Whey Protein Administration; Treatment of Cancer, Autoimmune and Infectious Diseases

Cancer is a group of diseases that are characterized by uncontrolled cell growth. The cancer cell is a transformed normal cell from any tissue that does not respond to growth regulation. Such transformed cell from any tissue has the potential to act as a stimulator of an immune response, either aggressive towards the cancer cell or immunosuppressive.

Kiessling et al. teach that progressive cancers have evolved distinct tumor escape mechanisms; hematological malignancies, brain tumors as well as the majority of solid tumors in their advanced stages display immunosuppression (Kiessling et al., 1999, Cancer Immunol. Immunother. 1999, 48(7): 353-62).

Kiessling et al. also teach that autoimmune and infectious diseases, too, have been connected to immune suppression (dysfunction) resulting in progressive disease. One of the underlying causes of autoimmune- and infection-associated immune regulatory dysfunction of the T lymphocytes is the intracellular signaling deficiency resulting from reduction of the TCR zeta chain expression. This dysfunction has been recorded in the autoimmune disease rheumatoid arthritis (RA) and in the infectious diseases leprosy and AIDS (Kiessling et al., 1999, above).

It is therefore plausible to postulate that a cancer treatment, which aims to enhance the host immune system, would have beneficial therapeutic effects.

Eason et al. teach that lifetime exposure to whey protein hydrolysate (WPH) in rat, relative to casein (CAS), decreased mammary tumor incidence and prolonged the appearance of tumors in methyl-N-nitrosourea (NMU)-treated female rats, with no corresponding effects on tumor multiplicity (Eason et al., Nutr. Cancer 2006, 55(2): 171-7).

However, Kennedy et al. and O'Dwyer et al. teach that GSH concentrations are high in tumor cells, giving them resistance to chemotherapeutic agents (Kennedy et al., Anticancer Res., 1995, 15: 2643-2649; O'Dwyer et al., Hematology/Onctology Clinics of North America 9(2) 383-396, 1995).

Similarly, Iantomasi et al. teach that the stimulation by PDGF of serum-starved NIH3T3 cells increases cellular GSH content, while no change in oxidized GSH content was measured (Iantomasi et al., Biochimica et Biophysica Acta—Molecular Cell Research, 1452, 3: December (1999), 303-312).

Accordingly, it remains unclear whether administration of substantially undenatured whey protein, which has been shown to elevate intracellular GSH (Kent et al., Toxicol. In Vitro 2003 February; 17(1): 27-33), would have beneficial therapeutic effects.

To date, few clinical human trials on whey and cancer have been conducted.

Kennedy et al. report a small 6 months trial conducted on five patients with metastatic carcinoma of the breast, one patient with pancreatic cancer, and one with liver cancer. In six patients, blood lymphocyte GSH was elevated initially, suggesting high tumor GSH levels. After completion of the study where the patients were administered a whey protein concentrate composition, two of the patients showed signs of tumor regression and a return of lymphocyte GSH levels to normal, while two patients showed signs of tumor stabilization without normalization of glutathione levels. Three patients had progression of disease and lymphocyte GSH levels increased from initial measurements.

From the conflicting results and small size of this study it remains unclear whether administration of undenatured whey protein composition alone has any therapeutic effect on human cancer. It also remains unclear whether some individuals may be substantially unresponsive to the undenatured whey protein administration due to inefficient assimilation of the undenatured whey protein composition.

### STATEMENT OF THE INVENTION

According to one aspect of the invention there is provided a novel composition and method of using such for treating a degenerative or an immune-related disease.

According to one aspect of the invention there is provided a method for treating a degenerative or an immune system-related disease, comprising oral administration of: (a) a therapeutically effective amount of a water composition having a O¹⁷ nuclear magnetic resonance (NMR) half-width value of between about 55 and about 85 Hz, wherein the water composition is substantially pathogen-, pyrogen-, heavy metal-, and virus-free; (b) a therapeutically effective amount of a substantially undenatured whey protein; and (c) a therapeutically effective amount of a sprouted grain composition comprising substantially undenatured digestive enzymes, wherein (a) and (b) are administered contemporaneously or sequentially in any order; wherein (c) is administered sequentially either: (i) before administration of both, or before administration of the first, of (a) and (b); or (ii) after administration of both, or after administration of the last, of (a) and (b); and wherein there is a therapeutically effective delay between sequential administration of (c) and of either or both (a) and (b).

In accordance with another aspect of the invention, there is provided a therapeutic composition, comprising digestive enzymes from sprouted grain, water having a O¹⁷ nuclear magnetic resonance (NMR) half-width value of between about 55 and about 85 Hz, wherein the water composition is pathogen-, pyrogen-, heavy metal-, and virus-free, and whey protein, each of which component may be provided in a single or in separate containers or commercial packages, and which further comprise instructions for use of the composition for treating a degenerative or an immune system-related disease.

Accordingly, the invention further provides a kit or a commercial package, comprising: (a) a therapeutically effective amount of a water composition having a O¹⁷ nuclear magnetic resonance (NMR) half-width value of between about 55 and about 85 Hz, wherein the water composition is substantially pathogen-, pyrogen-, heavy metal-, and virus-free; (b) a therapeutically effective amount of a substantially undenatured whey protein; and (c) a therapeutically effective amount of a sprouted grain composition comprising substantially undenatured digestive enzymes, for treating a degenerative or an immune system-related disease according to the method as described above.

The invention further provides use of (a) a therapeutically effective amount of a water composition having a O¹⁷ nuclear magnetic resonance (NMR) half-width value of between about 55 and about 85 Hz, wherein the water composition is substantially pathogen-, pyrogen-, heavy metal-, and virus-free; (b) a therapeutically effective amount of a substantially undenatured whey protein; and (c) a therapeutically effective amount of a sprouted grain composition comprising substantially undenatured digestive enzymes, wherein (a) and (b) are adapted for contemporaneous or sequential use in any order, wherein (c) is adapted for sequential use either: (i) before use of both, or before use of the first, of (a) and (b); or (ii) after use of both, or after use of the last, of (a) and (b); and wherein there is a therapeutically effective delay between sequential use of (c) and of either or both (a) and (b), for treating a degenerative or an immune system-related disease.

The invention further provides use of (a) a therapeutically effective amount of a water composition having a O¹⁷ nuclear magnetic resonance (NMR) half-width value of between about 55 and about 85 Hz, wherein the water composition is substantially pathogen-, pyrogen-, heavy metal-, and virus-free; (b) a therapeutically effective amount of a substantially undenatured whey protein; and (c) a therapeutically effective amount of a sprouted grain composition comprising substantially undenatured digestive enzymes, in the preparation of a medicament for treating a degenerative or an immune system-related disease.

In one embodiment, a therapeutic effective delay is one in which there is allowed between use or administration of the sprouted grain composition and use, absorption or administration of either or both the water composition and the whey protein, a sufficient time for obtaining an effective therapeutic result.

In one embodiment, when the sprouted grain composition is used or administered before use, absorption or administration of both or the first of the water composition and the whey protein, such therapeutic effective delay is at least 1 hour, or at least 1 hour and a half, or at least 2 hours, or at least 2 hours and a half, or at lest three hours.

In another embodiment, when the sprouted grain composition is used or administered after use, absorption or administration of both or of the last of the water composition and the whey protein, such therapeutic effective delay is at least 10 minutes, or at least 15 minutes, or at least 20 minutes, or at least 25 minutes, or at least 30 minutes.

In one embodiment, a degenerative or an immune system-related disease is selected from, but is not limited to, an auto-immune disease, cancer, a viral infection, i.e. sudden acute respiratory syndrome (SARS), hepatitis, HIV/AIDS, herpes, common cold, bacterial super infections, and the like.

### BRIEF DESCRIPTION OF THE DRAWINGS

FIG. 1. Effect of treatment with a composition comprising an embodiment of the invention on arterial blood pressure of HIV-infected patient.

FIG. 2. Effect of treatment with a composition comprising an embodiment of the invention on average body weight of HIV-infected patient.

FIG. 3. Graphical representation of the effect of treatment with a composition comprising an embodiment of the invention on average CD4 count of total HIV-infected patients in Kodjoviakope, Agou and Kouve.

FIG. 4. Graphical representation of the effect of treatment with a composition comprising an embodiment of the invention on average body weight of total HIV-infected patients in Kodjoviakope, Agou and Kouve.

FIGS. 5A to C. Graphical representation of the effect of treatment with a composition comprising an embodiment of the invention on Bio Impedance Alpha (BIA) parameters of HIV-infected patients in each of (A) Kodjoviakope, (B) Agou and (C) Kouve.

FIG. 6. Graphical representation of the effect of treatment with a composition comprising an embodiment of the invention on Bio Impedance alpha (BIA) parameters of total HIV-infected patients in Kodjoviakope, Agou and Kouve.

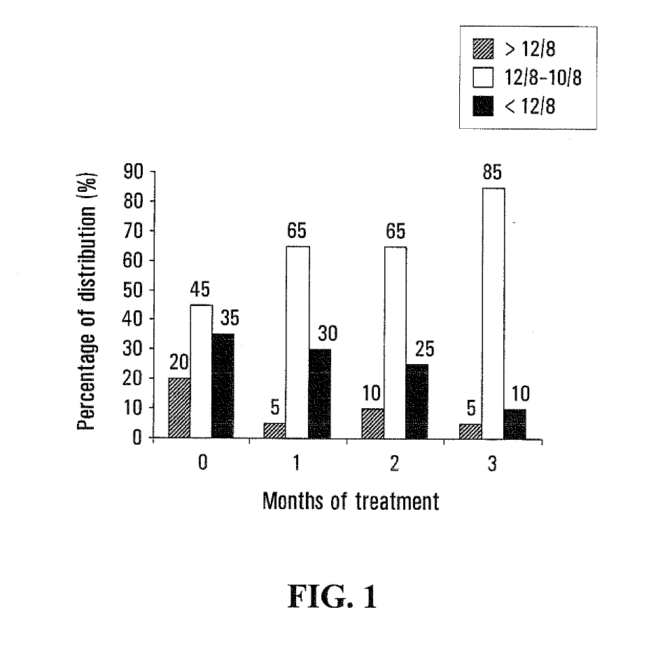

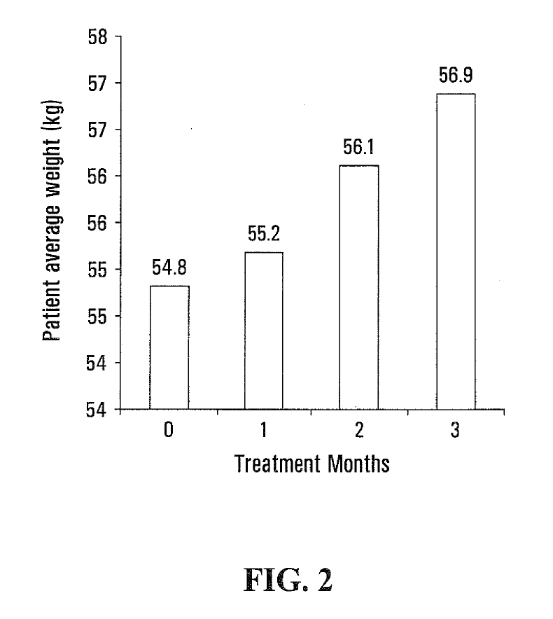

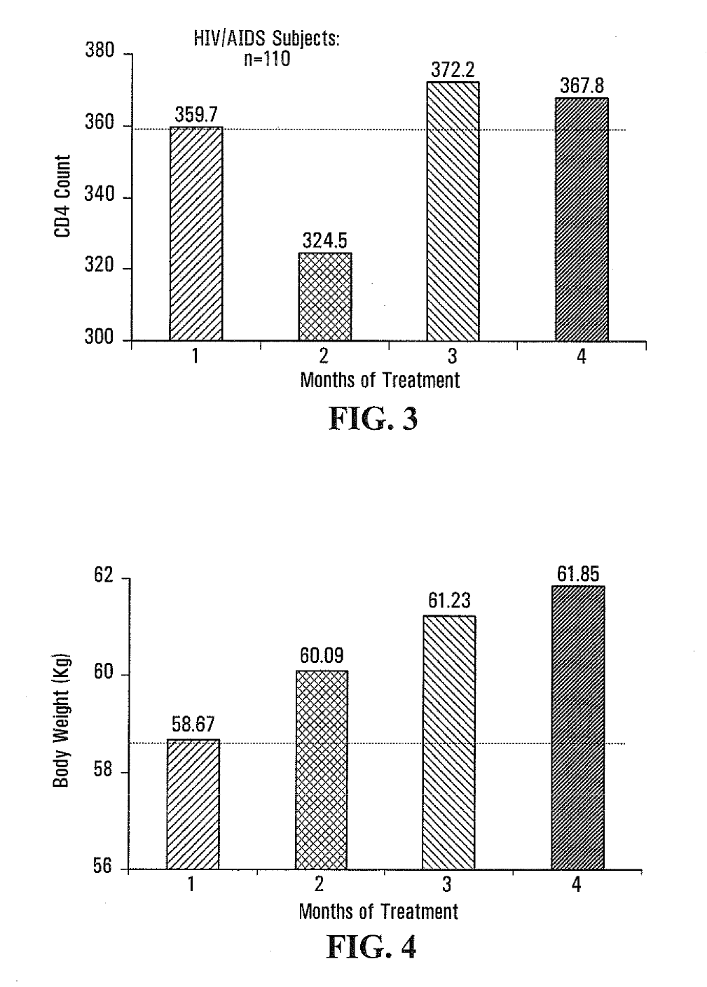

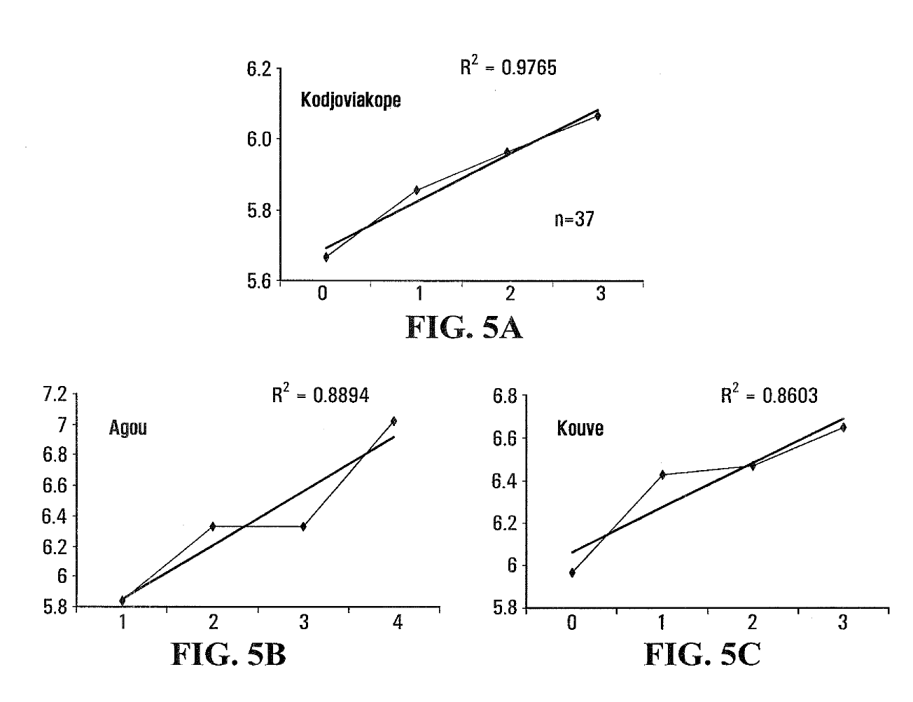

### DETAILED DESCRIPTION OF THE INVENTION

The inventors have discovered a composition and a method of using such for treating a degenerative or an immune system-related disease. In one embodiment, this desired result might be obtained by re-balancing the patient's homeostasis and/or enhancing the patient's immune system. In another embodiment, this desired result might be obtained by enhancing the patient's organism ability to effectively assimilate nutrients, by enhancing re-hydration of the organism, and/or the like.

The inventors have now demonstrated that, unexpectedly, the combination of a water composition, a sprouted grain composition which comprises digestive enzymes, and substantially undenatured whey protein therapeutically treats a degenerative or an immune-system related disease to an extent which was believed to be unexpected in view of the state of the art.

The combination of these three components provides novel and unexpected results that were not suggested or taught in the art.

### 1. Whey Protein

Whey is a complex composition made up of protein, lactose, fat and minerals. Whey is made up of many protein subfractions, such as: Beta-lactoglobulin, alpha-lactalbumin, immunoglobulins (IgGs), glycomacropeptides, bovine serum albumin (BSA) and minor peptides such as lactoperoxidases, lysozyme and lactoferrin.

Modern filtering technology has improved dramatically in the past decade, allowing separation of some of the highly bioactive peptides—such as glycomacropeptides, lactoferrin, lactoperoxidase, and the like—from whey.

Some of these subfractions are only found in very minute amounts in cow's milk, normally at less than one percent. For example, one of the most promising subfraction for preventing various diseases, improving immunity and overall health, lactoferrin makes up approximately 0.5% or less of whey protein derived from cow's milk (whereas human milk protein will contain up to 15% lactoferrin).

Whey protein has been used for treating cancer, treating HIV infection, improve immunity, reduce stress and lower cortisol, increase brain serotonin levels, improve_liver function in those suffering from certain forms of hepatitis, reduce blood pressure, and improve performance, and the like (Marshall, Altern. Med. Rev. Jun; 9(2): 136, 2004). Whey protein also has an exceptionally high biological value rating and an exceptionally high Branch Chain Amino Acid (BCAA) content.

Whey proteins in their natural substantial undenatured state (i.e. native conformational state) have biological activity (Bounos and Gould, Clin. Invest. Med. 14: 4; 296-309). Processing whey to remove the lactose, fats, etc. without losing protein biological activity takes special care.

Maintaining the substantial undenatured state of whey protein, or of at least one, or of at least two, or of at least three, or of at least four, of its subfraction is essential to the immune-modulating activity and/or for treating an immune system-related disease. The whey protein must be processed under low temperature, low acid conditions, other non-denaturing conditions known to those skilled in the art, or combinations thereof, as not to denature the whey protein.

Over the past few decades, whey protein powders have evolved several generations from low protein concentrates to very high protein isolates.

### 1.1. Whey Protein Concentrates (WPC)

First generation whey proteins contain as low as 30-40% protein (w/w of total product) and high amounts of lactose and fat. They are categorized as a whey concentrate. Modern concentrates now contain as high as 70-80% protein (w/w of total product) with reduced amounts of lactose. This is achieved through ultra-filtration processing, which removes lactose, thus elevating the concentration of protein and fat in the final product. A well-made concentrate is a high quality source of whey protein, though it will contain higher levels of lactose, ash, and fat then a whey protein isolate.

### 1.2. Whey Protein Isolates (WPI)

Isolates generally contain as much as 90-96% protein (w/w of total product). One advantage of WPI over WPC is that it contains more protein and less fat, lactose, and ash than concentrates on a gram for gram basis.

### 1.3. Ion Exchange

Running a whey concentrate through an ion exchange column makes ion exchange whey protein. However, due to the nature of the ion exchange process, some of the most valuable and health-promoting subfractions are likely selectively depleted, unless special care is taken or the ion exchange process improved.

1.4. Other Processing Techniques

With the array of more recent processing techniques used to make WPI—or pull out various subfractions from whey—such as Cross Flow Micro filtration (CFM™), ultra filtration (UF), micro filtration (MF), reverse osmosis (RO), dynamic membrane filtration (DMF), ion exchange chromatography, (IEC), electro-ultrafiltration (EU), radial flow chromatography (RFC) and nano filtration (NF), and others known to the skilled person in the art (such as those described in, for example, U.S. Pat. No. 7,018,665, 6,998,259, or 6,875,459), it is now possible to make very high grade and unique whey proteins.

The CFM processing method uses a low temperature micro filtration technique that allows for the production of very high protein contents (>90% protein w/w total product), the retention of important subfractions, extremely low fat and lactose contents, with virtually no undenatured proteins.

CFM is a natural, non-chemical process, which employs high tech ceramic filters, unlike ion exchange, which involves the use of chemical reagents such as hydrochloric acid and sodium hydroxide. CFM whey isolate also contains high amounts of calcium and low amounts of sodium.

### 1.5 Amino Acids

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, carboxyglutamate, and O-phosphoserine. Amino acid analogs refers to compounds that have the same basic chemical structure as a naturally occurring amino acid, i.e., an .alpha. carbon that is bound to a hydrogen, a carboxyl group, an amino group, and an R group, e.g., homoserine, norleucine, methionine sulfoxide, methionine, and methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

As to individual substitutions, deletions or additions that alter, add or delete a single amino acid, or a small percentage of amino acids from the whey protein of the invention and which create a “conservatively modified variant,” where the'alteration results in the substitution of an amino acid with a chemically similar amino acid, are also encompassed by the present invention. Conservative substitution tables providing functionally similar amino acids are well known in the art. Such conservatively modified variants are in addition to the invention.

The following groups each contain amino acids that are conservative substitutions for one another:
1) Alanine (A), Glycine (G);
2) Serine (S), Threonine (M;
3) Aspartic acid (D), Glutamic acid (E);
4) Asparagine (N), Glutamine (Q);
5) Cysteine (C), Methionine (M);
6) Arginine (E), Lysine (K), Histidine (H);
7) Isoleucine (I), Leucine (L), Valine (V); and
8) Phenylalanine (F), Tyrosine (Y), Tryptophan (W).
   (see, e.g., Creighton, Proteins, 1984, W. H. Freeman for a discussion of amino acid properties).
   2. Enzymes from Sprouted Grains

While raw vegetables and fruits have enzymes, they are low in concentration compared to sprouted seeds. The differences in enzyme concentration are quite significant.

There is from 10 to 100 times more enzymes in sprouted seeds than in vegetables or fruits depending on the enzyme and the seed that is being sprouted. Sprouted seeds are also a source of at least vitamins C, carotenoid, A, B vitamins, and minerals (Lintschinger et al., Plant Foods Hum. Nutr. 1997, 50(3): 223-37; Chavan and Kadam, Crit. Rev. Food Sci. Nutr. 1989, 28(5): 401-37; Lorenz, Crit. Rev. Food Sci. Nutr. 1980, 13(4): 353-85).

Sprouting of grains for a limited period causes increased activities of hydrolytic enzymes, improvement in the contents of certain essential amino acids, total sugars, and B-group vitamins, and a decrease in dry matter, starch, and anti-nutrients. The digestibility of storage proteins and starch are improved due to their partial hydrolysis during sprouting (Chavan and Kadam, Crit. Rev. Food Sci. Nutr. 1989, 28(5): 401-37).

### 2.1. Sprouted Grain Compositions

Sprouted seeds are germinated over at least two days, or at least three days, or at least four days, or at least five days, or at least six days, or at least seven days, or when enzyme activity in the seed reaches a maximum and the sprout is still relatively small. In some cases, after approximately the 5th day, enzyme content drops off markedly, as the sprout grows. Sprouts are grown long, like vegetables, and have very little enzymes compared to sprouted seeds. Several methods of culture and of obtaining sprouting are available to the person skilled in the art, such as, for example, those described in U.S. Pat. No. 5,802,965, 5,636,324, or 6,544,572.

The invention provides compositions and methods of treatment using sprouted grain compositions, in which a therapeutic dose of digestive enzymes from sprouted grain is orally administered in a pharmacologically acceptable formulation, e.g. to a patient or subject.

Accordingly, the invention also provides therapeutic compositions or kits comprising the digestive enzymes from sprouted grain and a pharmacologically acceptable excipient or carrier.

In one embodiment, such compositions or kits comprise undenatured digestive enzymes from sprouted grain in a therapeutically effective amount sufficient to re-balance an individual malnourished state, preferably an individual with an immune system-related disease. The therapeutic composition may be soluble in an aqueous solution at a physiologically acceptable pH or in another suitable form for oral administration, such as, but not limited to, a tablet form.

In accordance with the invention, there is provided a sprouted grain composition made out of edible sprouted grains which are rich in digestive enzymes.

In one embodiment, the sprouted grains-used in the present invention are selected from rye, kamut™, hemp, wheat, spelt, and the like, or combinations thereof.

### 3. Water Composition

In one aspect of the invention, there is provided a therapeutically effective amount of a water composition having a O¹⁷ nuclear magnetic resonance (NMR) half-width value of between about. 55 and about 85 Hz, wherein the water composition is pathogen-, pyrogen-, heavy metal-, and virus-free.

In one embodiment, said water composition comprises water having a reduced surface tension or water having a O¹⁷ nuclear magnetic resonance half-width value of between: about 55 and about 85 Hz, or about 56 and about 85, or about 57 and about 85, or about 58 and about 85, or about 59 and about 85, or about 60 and about 85, or about 61 and about 85, or about 62 and about 85, or about 63 and about 85, or about 64 and about 85, or about 65 and about 85, or about 55 and about 84, or about 55 and about 83, or about 55 and about 82, or about 55 and about 81, or about 55 and about 80, or about 55 and about 79, or about 55 and about 78, or about 55 and about 76, or about 55 and about 75 Hz.

In another embodiment, said water composition has a pH of between: about 3.5 and about 8.5, or about 4.0 and about 8.0, or about 4.5 and about 7.5, or about 5.0 and about 7.0, or about 4.0 and about 8.5, or about 4.5 and about 8.5, or about 5.0 and about 8.5, or about 5.5 and about 8.5, or about 6.5 and about 8.5, or about 7.0 and about 8.5.

For carrying the invention, one must make sure that the water is safe for human ingestion. In one embodiment, the water composition has less than about 20 mg per serving of silicate.

### 4. Definitions

A “therapeutically effective amount” refers to an amount effective, at dosages and for periods of time necessary, to achieve the desired therapeutic result, such as, but not limited to, increased absorption of nutriments, elevation of intracellular GSH levels, diminished viral titer, elevated white blood cell count, and the like known to those skilled in the art.

A “therapeutically effective amount” may vary according to factors such as, but not limited to, the disease state, age, sex, and weight of the individual, the ability of the compound to elicit a desired response in the individual, and the like known to those skilled in the art.

A “therapeutically effective amount” is also one in which any toxic or detrimental effects of the compound are outweighed by the therapeutically beneficial effects. For any particular subject, specific dosage regimens and delay between oral use or administration may be adjusted over time according to the individual need and the professional judgement of the person administering or supervising the administration of the compositions.

A “therapeutically effective delay” is one in which there is provided sufficient time to exclude significant detrimental effects between administration or use of either of the components of the invention.

A “therapeutically effective delay” is also one in which there is provided sufficient time between administration or use of the components of the invention to exclude significant detrimental interaction between the digestive enzymes, the whey protein and/or the clustered water composition of the invention.

As used herein “pharmaceutically acceptable carrier” or “excipient” includes any and all solvents, dispersion media, coatings, antibacterial and anti-fungal agents, isotonic and absorption delaying agents, and the like that are physiologically compatible. In one embodiment, the carrier is suitable for oral administration.

Pharmaceutically acceptable carriers also include sterile aqueous solutions or dispersions and sterile powders for the extemporaneous preparation of sterile dispersion solutions. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into each of the compositions.

Therapeutic compositions typically are sterile and/or stable under the conditions of manufacture and storage. The composition can be formulated as a powder, solution, microemulsion, liposome, tablet, or other ordered suitable structure. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Isotonic agents, for example, sugars, polyalcohols such as mannitol, sorbitol, or sodium chloride, or non-toxic agents may be included in the composition.

Prolonged absorption of the compositions can be brought about by including in the composition an agent which delays absorption, for example, monostearate salts and gelatin, and the like known to those skilled in the art.

Moreover, the composition'of the present invention can be administered in a time release formulation, for example in a composition which includes a slow release polymer, and the like known to those skilled in the art. The active compounds can be prepared with carriers that will protect the compound against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, polylactic acid and polylactic, polyglycolic copolymers (PLG), and the like known to those skilled in the art. Many methods for the preparation of such formulations are patented or generally known to those skilled in the art.

As used herein, “homeostasis” relates to the tendency of an organism or cell to regulate its internal conditions, such as the chemical composition of its body fluids, so as to maintain health and functioning, i.e. to maintain equilibrium and/or a balance. Where a disease or illness affects an individual, it will be understood that this individual would no longer have a balanced homeostasis.

Accordingly, as used herein a treatment to “re-balance homeostasis” relates to a treatment which affects the individual's organism so that internal conditions, such as the chemical composition of its body fluids, are affected in order to help the individual recover from the disease or illness.

As used herein “oral administration” comprises oral ingestion, sublingual administration, intra-buccal administration, and the like.

The following Examples are for illustrative purposes only and are not intended to limit the scope of the invention.

### EXAMPLES

### 1.1. Whey Protein

In one embodiment, the whey protein has an immunoenhancement activity and/or the whey protein restores the depleted or elevates the intracellular GSH level.

In another embodiment, it is the undenatured state of the whey protein which is important for the immunoenhancement activity and/or to restore the depleted or elevate the intracellular GSH level.

In yet another embodiment, it is the undenatured bovine serum albumin content and undenatured immunoglobulin content of the whey protein which is important for the immunoenhancement activity and/or to restore the depleted or elevate the intracellular GSH level.

In a further embodiment, it is the undenatured glycomacropeptide content which is important for the immunoenhancement activity and/or to restore the depleted or elevate the intracellular GSH level.

In yet a further embodiment, it is the undenatured lactoferrin content which is important for the immunoenhancement activity and/or to restore the depleted or elevate the intracellular GSH level.

In one further embodiment, the whey protein is in a powder form, a solution form, or a suitable form for oral administration.

In another yet further embodiment, the whey protein used in the invention is the proprietary GSH Complex™ whey (Cymcorp International, Inc.). GSH Complex™ comprises undenatured whey protein (at least 90%) with the following typical amino acid profile:

TABLE I
GSH Complex ™ typical amino acid profile

| Amino acid | Grams per 100 g of product |
|---|---|
| Alanine | 7.0 |
| Arginine | 1.8 |
| Aspartic acid | 9.3 |
| Cysteine/Cystine | 4.0 |
| Glutamic acid | 13.2 |
| Glycine | 2.6 |
| Histidine | 1.5 |
| Isoleucine | 5.0 |
| Leucine | 12.6 |
| Lysine | 8.9 |
| Methionine | 2.2 |
| Phenylalanine | 2.9 |
| Proline | 4.1 |
| Serine | 4.5 |
| Threonine | 4.9 |
| Tryptophane | 1.8 |
| Tyrosine | 2.7 |
| Valine | 5.2 |

GSH Complex™ has the following typical protein content:

TABLE II
GSH Complex ™ typical protein profile

| Protein subfraction | Relative fraction (%) |
|---|---|
| beta-lactoglobulin | 54% |
| alpha-lactalbulmin | 22% |
| glycomacropeptide | 21% |
| immunoglobulins | 2% |
| bovine serum albumine | 1% |
| lactoferrin | 0.49% |

In a further embodiment, there is provided a whey protein, comprising: whey protein isolate, whey protein concentrate, GSH Complex™, or a combination thereof.

### 1.2. Sprouted Grain Composition

In one embodiment, there is provided a sprouted grain composition which comprises undenatured digestive enzymes. In a further embodiment, there is provided a proprietary sprouted grain composition which comprises undenatured digestive enzymes: Triozyme™ (Cymcorp International, Inc.).

Triozyme™ is made from only organic certified whole grain sprouts typically consisting of sprouted spelt, rye and kamut™ in a typical 1:1:1 ratio. It comprises a full spectrum of vitamins, minerals, subtle and complex nutrients, and digestive enzymes. These digestive enzymes comprise amylase, lipase, protease, cellulase, and others. Triozyme™ may further comprise antioxidant enzymes, such as S.O.D., CoQ-10, catalase, and others. Triozyme™ may further comprise probiotic, such as lactobacillus and bifido, dietary fiber, essential fatty acids (EFA), amino acids, proteins, and omega 3 and 6.

Triozyme™ has the following typical enzyme profile when compared to non sprouted grains used in its manufacture:

TABLE III
TYPICAL ENZYME ANALYSIS OF TRIOZYME ™

| | Results | |
|---|---|---|
| | Non sprouted grain/ | |
| Analysis | Triozyme ™ | Unit |
| Alpha-Amylase | 0.63/73 | DU/g |
| Beta-Amylase | 0.63/250 | Unit/g |
| Lipase | 1.95/884 | LU/g |
| Protease | 0.009/27000 | HUT/g |
| S.O.D. | 0.00/87.7 | Unit/g |

The oral administration of Triozyme™ provides benefits selected from: help in digesting proteins, fats, sugars and fibre; enhancement of the assimilation of nutrients; balancing and/or correction of nutritional deficiencies; increase in vitality and energy; help in correcting constipation; and combinations thereof.

### 1.3. Water Composition

In one embodiment, there is provided a water composition comprising VIVO™ clustered water (described in U.S. Pat. Nos. 5,711,950 and 6,033,678) which is designed to restructure water into hexagonally organized bio-molecular clusters. A method of obtaining and using VIVO™ clustered water is detailed in U.S. Pat. Nos. 5,711,950 and 6,033,678.

This product can increase cellular hydration, enhance nutrition and oxygen delivery, restore cellular detoxification functions, and improve cellular electrical properties (Wang et al., Asia Pac. J. Clin. Nutr. 2004, 13 (Suppl): S128).

In one embodiment, VIVO™ clustered water is diluted in highly purified water or in distilled water with a resistance of: at least 2 Megaohms (Ω), or at least 2.5, or at least 3.0, or at least 3.5, or at least 4.0, or at least 4.5, or at least 5.0, or at least 5.5, or at least 6.0, or at least 6.5, or at least 7.0, or at least 7.5, or at least 8.0, or at least 8.5, or at least 9.0, or at least 9.5, or at least 10.0 Megaohms.

### 2. Clinical Trials

An embodiment of the novel combination of the invention and method of using such was assessed for its therapeutic efficiency. This clinical study is a double blind clinical randomized trial with placebo control, conducted for a period of one month, on half the sample. The target population comprises patients with HIV/AIDS that have been selected based on specific criteria from one urban zone and two rural zones in Togo, Africa.

### 2.1. Selection of HIV-Infected Patients

Sampling was randomized using exclusion criteria. The following formula was used for sampling:

$$N = \frac{Z {α}^{2} p \cdot q}{{i}^{2}}$$

### Where:

N=Number of subjects (host); and
Zα²=normal deviate (Z-score) corresponding to the risk a assessed.
In general, through convention, α=5%, where Zα=1.96.
Therefore, we accept to take the risk five times out of 100.
p=approximate prevalence 5% of the factor studied;
q=complement of p (q=100−p); and
i=precision, generally 5%.
Given the variation in seroprevalence rates throughout the regions, the maximum was used, which is equivalent to 5% (p).

Thus, based on the calculations, N=82 after having adjusted for the loss-of patients during the study period. This number was brought to 115 to further minimize sampling errors.

TABLE IV
Distribution of Subjects

| SITE | WOMEN | MEN | TOTAL |
|---|---|---|---|
| 1. Centre de Santé | 20 | 19 | 39 |
| KODJOVIAKOPE | | | |
| 2. Centre Médico-social | 19 | 19 | 38 |
| KOUVE | | | |
| 3. Hôpital BETHESDA | 19 | 19 | 38 |
| d'AGOU | | | |
| TOTAL | 58 | 57 | 115 |

In total, one hundred and fifteen (115) sero-positive (HIV-infected) subjects were tested in the clinical study. Gender has been taken into account in recruiting statistical units; there are thus 58 women and 57 men in the study.

HIV-infected patients aged 18 to 45 years have been selected based on having CD4 rate between about 150 and about 300, with an approximate weight of about 63 kg, in each zones (urban and rural).

The following selection exclusion criteria was used:
Patients with liver disease;
alcoholic patients;
patients with medical dependency;
patients being treated with anti-AIDS pharmaceutical drugs (ARV);
patients with an allergy to whey; and
patients on immuno-suppressant therapy.

### 2.2. Experimental Protocol

The following experimental protocol was used.

### Month 1

Group No. 1

This group received placebos, whose physical properties are the same as the tested products, except that they are not biologically active.

### Group No. 2

This group received the following biologically active products:
Substantially undenatured whey protein (GSH complex™) 20 g×twice a day for one (1) month;
500 ml of a water composition comprising clustered water VIVO™ to prepare the whey protein; and
2 grams of enzyme complex (Triozyme™) twice a day with meals.
Months 2 and 3

All patients from both groups are put on the biologically active products.

Total clinical assay period: Three (3) months. After one (1) month, the placebo group was put on the biologically active products because their biological parameters indicated general health degeneration at that time. Accordingly, for ethical and humanitarian reasons, the placebo was replaced after one (1) month of clinical trials.

### 2.3. Detailed Description of the Experimental Protocol

Each participant was administered, twice daily, 12 ounces of the water composition to which was added twenty grams of GSH Complex™, freshly prepared.

Each participant was also administered chewable digestive enzyme tablets (Triozyme™ grain composition) that were to be taken before and after each meal. Meals had to be taken within at least 30 minutes after taking the GSH Complex™ and water composition.

### 2.4. Data Gathering

Amongst the data usually gathered during clinical trials, known to those skilled in the art, the following measures were taken:
Symptoms present;
Anthropometric measurements, such as body weight (initial and monthly), temperature (in degrees), BP, Conjunctiva, skin and appendages, types of opportunistic infections, and the like; and
Biological constants, such as blood test (initial and monthly), HIV test, CD4, Transaminases, FBC, Creatinine, Glycemic index, Triglycerides, Cholesterol, and the like.

Data was entered using EPI Data (Lauritsen J M. (Ed.) EpiData Data Entry, Data Management and basic Statistical Analysis System. Odense Denmark, EpiData Association, 2000-2006) and analyzed using SPSS™ 11 software (SSPS Inc.).

The following follow-up indicators were used:
Loss rate of HIV-infected patients; and
Percentage of HIV-infected patients that remained stable or which had improved their health on the basis of:
   (i) clinical elements, i.e. general condition weight of appendages, hue of conjunctiva, frequency of opportunistic infections, anorexia, and the like; and
   (ii) paraclinical elements, i.e. CD4 hemogram, and other required tests known to those skilled in the art.
      The follow-ups were carried out monthly by meeting with the stakeholders in order to make adjustments when appropriate.

### 2.5. Results and Data Obtained

The following tables represent the biological parameters observed during clinical trials with an embodiment of the invention and are for illustrative purposes only.

TABLE V
Values obtained in Kodjoviakope

| Biological | | Months of treatment | | | |
|---|---|---|---|---|---|
| parameter | Values | Month 0 | Month 1 | Month 2 | Month 3 |
| Weight (kg) | average | 66.8 | 68.3 | 69.5025 | 70.4 |
| | median | 65 | 66 | 66.5 | 67.5 |
| | Standard deviation | 18.5 | 17.3 | 18.3 | 18.2 |
| TEMPERATURE | average | 37.37 | 37.185 | 37.38 | 37.65 |
| (° C.) | median | 37.5 | 37 | 37.5 | 38 |
| | Standard deviation | 0.4 | 0.2 | 0.3 | 0.5 |
| CD4 count | average | 347.15 | 327.5 | 327.55 | 359.25 |
| | median | 313.5 | 286.5 | 279 | 290.5 |
| | Standard deviation | 154.4 | 177.3 | 160.1 | 180.0 |
| NFS Red blood | average | 4434500 | 4380500 | 4398000 | 4409500 |
| cell count | median | 4375000 | 4275000 | 4370000 | 4370000 |
| | Standard deviation | 934 009.3 | 852 102.5 | 933 632.4 | 927 217.0 |
| NFS haemoglobin | average | 11.61 | 11.33 | 11.17 | 11.11 |
| count | median | 11.25 | 11.05 | 11.3 | 11.4 |
| | Standard deviation | 2.1 | 1.9 | 1.8 | 1.4 |
| NFS leucocytes | average | 4770 | 4620 | 4975 | 4855 |
| count | median | 4550 | 4250 | 4350 | 4400 |
| | Standard deviation | 1 249.0 | 1 346.2 | 2 108.1 | 1 621.4 |
| NFS % | average | 55.45 | 56.55 | 49.9 | 57 |
| lymphocytes | median | 57 | 56.5 | 52.5 | 57.5 |
| | Standard deviation | 9.4 | 9.0 | 8.3 | 10.7 |
| Transaminases | average | 50.4 | 44.75 | 44.45 | 40.8 |
| SGOT | median | 45 | 41.5 | 41 | 37.5 |
| | Standard deviation | 32.2 | 25.4 | 20.4 | 13.4 |
| Transaminases | average | 31.15 | 28.9 | 37.5 | 30.8 |
| SGPT | median | 29.5 | 24.5 | 26.5 | 29 |
| | Standard deviation | 13.3 | 18.0 | 28.9 | 16.4 |
| Glycemic index | average | 0.959 | 0.952 | 0.8905 | 1.31 |
| | median | 0.92 | 0.975 | 0.89 | 0.875 |
| | Standard deviation | 0.3 | 0.1 | 0.1 | 1.8 |
| Triglycerides | average | 0.97 | 0.9065 | 0.8475 | 0.77 |
| | median | 0.725 | 0.8 | 0.765 | 0.725 |
| | Standard deviation | 0.9 | 0.5 | 0.4 | 0.3 |
| Cholesterol | average | 1.6175 | 1.5965 | 1.554 | 1.5115 |
| | median | 1.505 | 1.5 | 1.595 | 1.555 |
| | Standard deviation | 0.6 | 0.6 | 0.3 | 0.3 |
| Creatinine | average | 9.7 | 9.9 | 9.25 | 9.1 |
| | median | 10 | 9.5 | 8.5 | 9 |
| | Standard deviation | 1.2 | 2.4 | 2.5 | 1.3 |

TABLE VI
Values obtained in Agou after 3 months of treatment

| Biological | | Months of treatment | | | |
|---|---|---|---|---|---|
| parameter | Values | Month 0 | Month 1 | Month 2 | Month 3 |
| Weight (kg) | average | 54.8 | 55.2 | 56.1 | 56.9 |
| | median | 55.0 | 55.0 | 56.0 | 57.0 |
| | Standard deviation | 10.8 | 10.6 | 10.9 | 10.6 |
| TEMPERATURE | average | 36.7 | 37.0 | 36.9 | 36.8 |
| (° C.) | median | 36.8 | 37.0 | 37.0 | 37.0 |
| | Standard deviation | 0.5 | 0.6 | 0.7 | 0.5 |
| Arterial blood | average | 12.7 | 12.0 | 12.2 | 12.5 |
| pressure | median | 12.6 | 11.5 | 11.5 | 12.6 |
| | Standard deviation | 2.1 | 2.3 | 2.7 | 2.1 |
| CD 4 Count | average | 420 | 345 | 458 | 374 |
| | median | 42.0 | 32.0 | 36.0 | 34.0 |
| | Standard deviation | 13.7 | 10.6 | 31.9 | 15.3 |
| NFS Red blood | average | 3 695 588.2 | 3 465 823.5 | 3 173 294.1 | 3 533 823.5 |
| cell count | median | 3 700 000.0 | 3 461 000.0 | 3 346 000.0 | 3 585 000.0 |
| | Standard deviation | 903 223.2 | 553 629.2 | 1 028 878.7 | 723 631.1 |
| NFS haemoglobin | average | 11.8 | 11.0 | 9.8 | 11.2 |
| count | median | 11.5 | 11.3 | 10.2 | 11.5 |
| | Standard deviation | 2.6 | 1.8 | 3.4 | 2.5 |
| NFS leucocytes | average | 4 582.4 | 4 758.8 | 4 158.8 | 4 247.1 |
| count | median | 4 200.0 | 4 600.0 | 4 100.0 | 4 300.0 |
| | Standard deviation | 1 933.7 | 1 749.7 | 1 741.4 | 649.2 |
| NFS % | average | 49.5 | 42.9 | 45.6 | 51.9 |
| lymphocytes | median | 48.0 | 40.0 | 48.0 | 52.0 |
| | Standard deviation | 13.4 | 12.4 | 17.8 | 10.6 |
| TRANSAMINASES | average | 30.4 | 24.4 | 32.4 | 24.1 |
| SGOT (UI/l) | median | 20.0 | 24.0 | 22.5 | 23.0 |
| | Standard deviation | 17.9 | 8.8 | 23.2 | 7.8 |
| TRANSAMINASES | average | 436.2 | 337.6 | 389.0 | 378.1 |
| SGPT (UI/l) | median | 345.0 | 267.0 | 292.0 | 306.0 |
| | Standard deviation | 291.3 | 261.6 | 296.2 | 291.0 |
| Glycemic index | average | 0.6 | 0.6 | 0.7 | 0.7 |
| | median | 0.6 | 0.6 | 0.7 | 0.7 |
| | Standard deviation | 0.1 | 0.1 | 0.1 | 0.1 |
| Triglycerides | average | 1.0 | 1.0 | 0.6 | 0.8 |
| | median | 0.7 | 0.8 | 0.6 | 0.7 |
| | Standard deviation | 0.5 | 0.3 | 0.3 | 0.4 |
| Cholesterol | average | 1.0 | 0.8 | 1.1 | 0.9 |
| | median | 0.9 | 0.8 | 1.1 | 0.8 |
| | Standard deviation | 0.3 | 0.3 | 0.3 | 0.3 |
| Creatinine | average | 9.4 | 11.3 | 9.2 | 8.4 |
| | median | 8.7 | 8.2 | 8.2 | 7.9 |
| | Standard deviation | 2.2 | 8.9 | 2.5 | 2.1 |

Results from Kodjoviakope

The following is based on the results reported in the previous Tables and appended Figures.

After three months of treatment, the patient's temperature remains within normal values, i.e. from 37.4±0.4 to 37.7±0.5° C. Patient average weight elevates from 66.8±18.5 kg to 70.4±18.2 kg. After three months of treatment, 85% of patients recover normal blood pressure (120/80-100/80), whereas 45% had such normal blood pressure prior to the treatment. Accordingly, these data suggest that following treatment there is a general trend towards a re-balancing of health-indicating parameters.

After three months of treatment, the average CD4 count elevates from 347 to 359. However, the inventors observed a slight decrease in red blood cells through a decrease in average haemoglobin count (11.6% to 11.1%), which may be due to hemodilution due to enhanced hydration. Lymphocytes average count increased from 55.4% to 57%. Accordingly, these data suggest that following treatment, there is a general trend towards an enhancement of the patient's immune system.

After three months of treatment, average serum glutamic oxalacetic transaminase (SGOT) and average serum glutamic pyruvic transaminase (SGPT) diminish from 50.4 UI/l to 40.8 UI/l and from 31.15 to 30.8 UI/l, respectively. Average glycemic index diminishes from 0.9 g/l to 0.87 g/l. Average cholesterol values also diminish from 1.6 g/l to 1.5 g/l. Accordingly, these data suggest that following treatment there is a general trend towards re-balancing (normalisation) of the hepatic system.

After three months of treatment, average creatinine diminishes from 9.7 mg/l to 9.1 mg/l, but remains within normal values (7 mg/l-12 mg/l). Accordingly, these data suggest that the composition used, which is an embodiment of the invention, is not toxic to the renal system.

Results from Agou

The following is based on the results reported in the previous Tables and appended Figures. It is noteworthy that in the rural region of Agou, the nutritional habits include food intake which is high in carbohydrates and poor in proteins.

After three months of treatment, average weight elevates from 54.8±10.8 kg to 56.9±10.6 kg. The patient's temperature remains within the normal values of 36.7° to 36.8° C. Accordingly, these data suggest that following treatment there is a general trend towards re-balancing of health-indicating parameters.

After three months of treatment, the average CD4 count varies from 420 to 458 and then to 374. The inventors observed a slight decrease in red blood cells through a decrease in average haemoglobin count from 11.8% to 11.1%, as well as a slight decrease in leukocyte count from 4582 to 4247. The average lymphocyte count elevates from 49.9% to 51.9%. Accordingly, these data suggest that following treatment there is a slight enhancement of the patient's immune system.

After three months of treatment, average SGOT and average SGPT diminish from 30.4 UI/l to 24.1 UI/l and from 436.2 to 378.1 UI/l, respectively. Average glycemic index diminishes from 0.9 g/l to 0.87 g/l. Average cholesterol diminishes from 1 g/l to 0.9 g/l while average triglycerides remain within the normal values of 0.6 g/l-0.7 g/l. Accordingly, these data suggest that following treatment there is a general trend towards re-balancing (normalisation) of the hepatic system.

After three months of treatment, average creatinine diminishes from 9.4 mg/l to 8.4 mg/l, but remains within normal values (7 mg/l-12 mg/l). Accordingly, these data suggest that the composition used, which is an embodiment of the invention, is not toxic to the renal system.

### BIA Parameters

BIA provides a reliable estimate of total body water under most conditions. It can be a useful technique for body composition analysis in healthy individuals and in those with a number of chronic conditions such as mild-to-moderate obesity, diabetes mellitus, and other medical conditions in which major disturbances of water distribution are not prominent (NIH, American Journal of Clinical Nutrition 64, 524S-532S; and Sun et al., American Journal of Clinical Nutrition, 77: 331-340, 2003).

BIA has been shown to have a higher impact on prognostic of HIV-infected patients than a measurement of CD4 count (Ott et al., J. Acquir. Immune Defic. Syndr. Hum. Retrovirol. 1995, 1; 9(1): 20-5; Shwenk et al., Am. J. Clin. Nutr. 2000; 72(2): 496-501; and Shah et al., J. Nutr. 2001; 131(11): 2843-7).

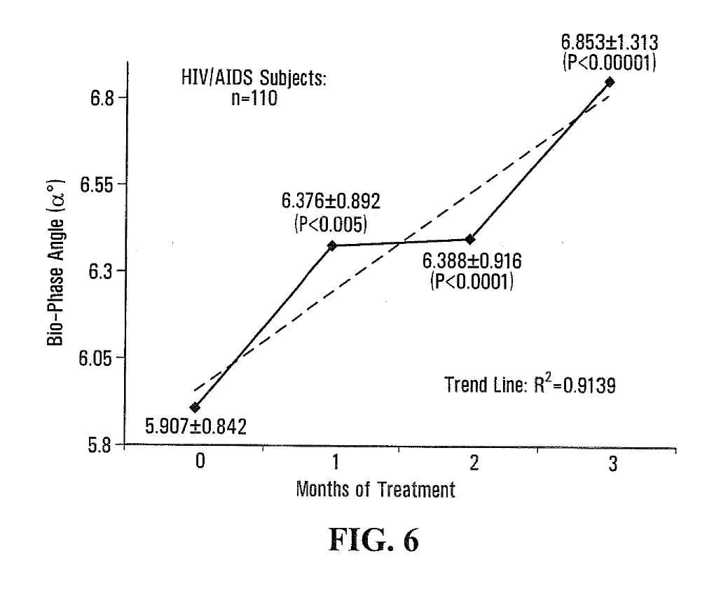

The BIA parameters were shown to be statistically significant with the Student t-test (see FIG. 6). These results suggest that the patients after only three (3) months of treatment have a clear indication that the organism is recovering from the disease; thus suggesting an amelioration of the patient prognostic.

Therefore, the biological parameters measured suggest that the administered treatment, which is an embodiment of the invention, has general health benefits for the patients. Such benefits are, but not limited to, an enhancement of the immune system, a normalisation of hepatic and renal function, and general recovery from the disease.

All references cited supra and infra are incorporated herein in their entirety by reference. In this patent application, the term patient is equivalent to the following terms: host, subject, individuals, and the like.

The foregoing is considered as illustrative only of the principles of the invention. Further, since numerous modifications and changes will readily occur to those skilled in the art, it is not desired to limit the invention to the exact examples and embodiments shown and described, and accordingly, all suitable modifications and equivalents may be resorted to, falling within the scope of the invention.

1-22. (canceled)

23. A nutritional composition useful for treating an HIV-seropositive individual so as to increase his bioelectrical impedance analysis (BIA), comprising:
(a) a water composition having an O¹⁷ nuclear magnetic resonance (NMR) half width value of between about 55 and about 85 Hz, wherein the water composition is substantially pathogen-, pyrogen, heavy metal-, and virus-free;
(b) a substantially undenatured whey protein; and
(c) a sprouted grain composition.

24. A nutritional composition according to claim 23, wherein the whey protein comprises substantially undenatured whey protein isolate.

25. A nutritional composition according to claim 23, wherein the whey protein has the following amino acid grams per 100 grams of whey protein:

| | |
|---|---|
| Alanine | 7.0 |
| Arginine | 1.8 |
| Aspartic acid | 9.3 |
| Cysteine/Cystine | 4.0 |
| Glutamic acid | 13.2 |
| Glycine | 2.6 |
| Histidine | 1.5 |
| Isoleucine | 5.0 |
| Leucine | 12.6 |
| Lysine | 8.9 |
| Methionine | 2.2 |
| Phenylalanine | 2.9 |
| Proline | 4.1 |
| Serine | 4.5 |
| Threonine | 4.9 |
| Tryptophane | 1.8 |
| Tyrosine | 2.7 |
| Valine | 5.2. |

26. A nutritional composition according to claim 23, wherein the whey protein has the following protein relative fraction:

| | |
|---|---|
| beta-lactoglobulin | 54% |
| alpha-lactalbulmin | 22% |
| glycomacropeptide | 21% |
| immunoglobulins | 2% |
| bovine serum albumine | 1% |
| lactoferrin | 0.49%. |

27. A nutritional composition according to claim 23, wherein the water composition comprises a first clustered water diluted in a second water selected from highly purified water, distilled water, and water having a reduced surface tension, wherein the second water has a resistance value of at least 2 Megaohms.

28. A nutritional composition according to claim 23, wherein the water composition has a pH of between about 3.5 and about 8.5.

29. A nutritional composition according to claim 23, wherein the sprouted grain composition comprises sprouted rye, kamut, hemp, wheat, spelt, or any combinations thereof.

30. A nutritional composition according to claim 23, wherein:
the water composition comprises a first clustered water diluted in a second water selected from highly purified water, distilled water, and water having a reduced surface tension, wherein the second water has a resistance value of at least 2 Megaohms;
the whey protein has the following protein relative fraction:

| | |
|---|---|
| beta-lactoglobulin | 54% |
| alpha-lactalbulmin | 22% |
| glycomacropeptide | 21% |
| immunoglobulins | 2% |
| bovine serum albumine | 1% |
| lactoferrin | 0.49%; and |
| | 52% |

the sprouted grain composition comprises sprouted rye, kamut, hemp, wheat and spelt.

31. A method of treating an HIV-seropositive individual so as to increase his bioelectrical impedance analysis (BIA), comprising oral administration to the HIV-seropositive individual of:
(a) a water composition having an O¹⁷ nuclear magnetic resonance (NMR) half-width value of between about 55 and about 85 Hz, wherein the water composition is substantially pathogen-, pyrogen-, heavy metal-, and virus-free;
(b) a substantially undenatured whey protein; and
(c) a sprouted grain composition,
wherein (a) and (b) are administered contemporaneously or sequentially in any order; and
wherein there is a delay between sequential administration of (c) and of either or both (a) and (b) that is sufficient to prevent degradation of the whey protein by the sprouted grain composition.

32. A method according to claim 31, wherein the whey protein comprises substantially undenatured whey protein isolate.

33. A method according to claim 31, wherein the whey protein has the following amino acid grams per 100 grams of whey protein:

| | |
|---|---|
| Alanine | 7.0 |
| Arginine | 1.8 |
| Aspartic acid | 9.3 |
| Cysteine/Cystine | 4.0 |
| Glutamic acid | 13.2 |
| Glycine | 2.6 |
| Histidine | 1.5 |
| Isoleucine | 5.0 |
| Leucine | 12.6 |
| Lysine | 8.9 |
| Methionine | 2.2 |
| Phenylalanine | 2.9 |
| Proline | 4.1 |
| Serine | 4.5 |
| Threonine | 4.9 |
| Tryptophane | 1.8 |
| Tyrosine | 2.7 |
| Valine | 5.2. |

34. A method according to claim 31, wherein the whey protein has the following protein subfraction relative fraction:

| | |
|---|---|
| beta-lactoglobulin | 54% |
| alpha-lactalbulmin | 22% |
| glycomacropeptide | 21% |
| immunoglobulins | 2% |
| bovine serum albumine | 1% |
| lactoferrin | 0.49%. |

35. A method according to claim 31, wherein the water composition comprises a first clustered water diluted in a second water selected from highly purified water, distilled water, and water having a reduced surface tension, wherein the second water has a resistance value of at least 2 Megaohms.

36. A method according to claim 31, wherein the water composition has a pH of between about 3.5 and about 8.5.

37. A method according to claim 31, wherein the sprouted grain composition comprises sprouted rye, kamut, hemp, wheat, spelt, or any combinations thereof.

38. A method according to claim 31, wherein:
the water composition comprises a first clustered water diluted in a second water selected from highly purified water, distilled water, and water having a reduced surface tension, wherein the second water has a resistance value of at least 2 Megaohms;
the whey protein has the following protein relative fraction:

| | |
|---|---|
| beta-lactoglobulin | 54% |
| alpha-lactalbulmin | 22% |
| glycomacropeptide | 21% |
| immunoglobulins | 2% |
| bovine serum albumine | 1% |
| lactoferrin | 0.49%; and |

the sprouted grain composition comprises sprouted rye, kamut, hemp, wheat and spelt.
